(19) United States
(12) Patent Application Publication
(10) Pub. No.: US 2005/0245539 A1
(43) Pub. Date: Nov. 3, 2005

(54) Pharmaceutical compositions for the treatment of sexual disorders II

(75) Inventors: Klaus Mendla, Biberach (DE); Thomas Friedl, Ochsenhausen (DE); Wolfram Eisenreich, Ulm (DE); Robert Pyke, New Fairfield, CT (US)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignees: Boehringer Ingelheim International GmbH, Ingelheim (DE); Boehringer Ingelheim Pharmaceuticals, Inc., Ridgefield, CT (US)

(21) Appl. No.: 11/110,449

(22) Filed: Apr. 20, 2005

Related U.S. Application Data
(60) Provisional application No. 60/564,662, filed on Apr. 22, 2004.
(60) Provisional application No. 60/631,800, filed on Nov. 30, 2004.

(51) Int. Cl. A61K 31/496; A61K 31/557

(52) U.S. Cl. 514/254.06; 514/573

(57) ABSTRACT

The invention relates to new pharmaceutical compositions for the treatment of sexual disorders and methods for the preparation thereof. In a preferred embodiment, the instant invention is directed to pharmaceutical combinations comprising flibanserin as one active ingredient in combination with at least one additional active ingredient for the treatment of sexual disorders and methods for the preparation thereof.

### RELATED APPLICATIONS

This application claims benefit to U.S. Provisional Application Ser. No. 60/564,662 filed Apr. 22, 2004 and U.S. Provisional Application Ser. No. 60/631,800 filed Nov. 30, 2004.

### BACKGROUND OF THE INVENTION

The invention relates to new pharmaceutical compositions for the treatment of sexual disorders and methods for the preparation thereof. In a preferred embodiment, the instant invention is directed to pharmaceutical combinations comprising flibanserin as one active ingredient in combination with at least one additional active ingredient for the treatment of sexual disorders and methods for the preparation thereof.

The invention relates to new pharmaceutical compositions for the treatment of sexual disorders and methods for the preparation thereof. In a preferred embodiment, the instant invention is directed to pharmaceutical combinations comprising a therapeutically effective amount of flibanserin 1 as one active ingredient in combination with a therapeutically effective amount of at least one additional active ingredient 2 for the treatment of sexual disorders and methods for the preparation thereof.

The compound 1-[2-(4-(3-trifluoromethyl-phenyl)piperazin-1-yl)ethyl]-2,3-dihydro-1H-benzimidazol-2-one (flibanserin) is disclosed in form of its hydrochloride in European Patent Application EP-A-526434 and has the following chemical structure:

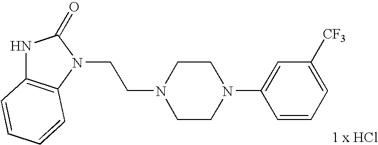

Flibanserin shows affinity for the 5-HT_{1A} and 5-HT₂-receptor. It is therefore a promising therapeutic agent for the treatment of a variety of diseases, for instance depression, schizophrenia, Parkinson, anxiety, sleep disturbances, sexual and mental disorders and age associated memory impairment.

### DETAILED DESCRIPTION OF THE INVENTION

A preferred embodiment of the invention is directed to pharmaceutical compositions comprising a therapeutically effective amount of flibanserin 1 in combination with a therapeutically effective amount of one or more, preferably one active ingredient 2 selected from the group consisting of melanocortin agonists, prostaglandin E1 agonists, elevators of cyclic guanosine 3′,5′-monophosphate (cGMP) (preferably PDE V inhibitors), 5-HT-1A agonists, dopamine agonists, dopamine D4 antagonist, 5-HT-2A/C antagonists, selective androgen receptor modulators (SARMs), selective estrogen receptor modulators (SERMs), estrogens, androgens and α-adrenergic receptor antagonists.

The compositions according to the invention may contain flibanserin 1 and the one or more additional active ingredient 2 in a single formulation or in separate formulations. If flibanserin and the one or more additional active ingredient are present in separate formulations these separate formulations may be administered simultaneously or sequentially.

A preferred embodiment according to the invention is directed to pharmaceutical compositions comprising a therapeutically effective amount of flibanserin 1 and a therapeutically effective amount of one or more, preferably one melanocortin agonist 2a, optionally in combination with a pharmaceutically acceptable excipient. Examples of suitable melanocortin agonists include PT-141, MCL-0129, PG-917, and Ro-27-3225, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one prostaglandin E1 agonist 2b, optionally in combination with a pharmaceutically acceptable excipient. Examples of suitable prostaglandin E1 agonists, include ornoprostil, limaprost, alprostadil, gemeprost, liprostin, NMI-775, prostaglandin E (PGE-1), papaverine, dioxyline, ethaverine, phentolamine, prazosin, minoxidil, nitroglycerin, alpha blockers, nitric oxide donors, and peptides (e. g., VIP), optionally in form of the pharmaceutically acceptable salts, in form of the hydrates and/or solvates and optionally in the form of the individual optical isomers, mixtures of the individual enantiomers or racemates thereof.

Preferred compounds 2b include ornoprostil, limaprost, alprostadil, gemeprost, liprostin and NMI-775, from which ornoprostil, limaprost and alprostadil are particularly preferred, optionally in form of the pharmaceutically acceptable salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one elevator of cGMP 2c, preferably a cGMP phosphodiesterase (cGMP PDE) inhibitor, more preferably a selective PDE V inhibitor, optionally in combination with a pharmaceutically acceptable excipient. Examples of elevators of cGMP, in particular examples for suitable PDE V inhibitors include vardenafil, sildenafil, tadalafil, NCX-911, Sch-444877, FR-229934, 4-bromo-5-(pyridylmethylamino)-6-[3-(4-chlorophnyl)-propoxy]-3(2H)pyridazinone, 1-[4-(1,3-benzodioxol-5-ylmethyl)amiono]-6-chloro-2-[quinozolinyl]-4-piperidine-carboxylic acid, monosodium salt, (+)-cis-5,6a,7,9,9,9a-hexahydro-2-[4-(trifluoromethyl)-phenylmethyl-5-methyl-cyclopent-4,5]imidazo[2,1-b]purin-4(3H)one, furaziocillin, cis-2-hexyl-5-methyl-3,4,5,6a,7,8,9,9a-octahydrocyclopent[4,5]-imidazo[2,1-b]purin-4-one, 3-acetyl-1-(2-chlorobenzyl)-2 propylindole-6-carboxylate, 3-acetyl-1-(2-chlorobenzyl)-2-propylindole-6-carboxylate, 4-bromo-5-(3-pyridylmethylamino)-6-(3-(4-chlorophenyl) propoxy)-3-(2H)pyridazinone, 1-methyl-5(5-morpholinoacetyl-2-n-propoxyphenyl)-3-n-propyl-1,6-dihydro-7H-pyrazolo(4,3-d)pyrimidin-7-one, 1-[4-[(1,3-benzodioxol-5- ylmethyl)amino]-6-chloro-2-quinazolinyl}-4-piperidinecarboxylic acid, monosodium salt, GF-196960, E-8010, E-4010, Bay-38-3045, Bay-38-9456, FR226807, Sch-51866, 5-(2-ethoxy-5-morpholinoacetylphenyl)-1-methyl-3-n-propyl-1,6-dihydro-7H-pyrazolo[4,3-d]pyrimidin-7-one, 3-ethyl-5-[5-(4-ethylpiperazin-1-ylsulfonyl)-2-n-propoxyphenyl]-2-(pyridin-2-yl)methyl-2,6-dihydro-7H-pyrazolo[4,3-d]pyrimidin-7-one, 3-ethyl-5-[5-(4-ethylpiperazin-1-ylsulfonyl)-2-(2methoxyethoxy)pyridin-3-yl]-2-(pyridin-2-yl)methyl-2,6-dihydro-7H-pyrazolo[4,3-d]pyrimidin-7-one, (+)-3-ethyl-5-[5-(4-ethylpieperazin-1-ylsulfonyl)-2-(2-methoxy-1(R)-methylethoxy)pyridin-3-yl]-2-methyl-2,6-dihydro-7H-pyrazolo[4,3-d]pyrimidin-7-one, 5-[2-ethoxy-5-(4-ethylpiperazin-1-ylsulfonyl)pyridin-3-yl]-3-ethyl-2-[2-methoxyethyl]-2,6-dihydro-7H-pyrazolo[4,3-d]pyrimidin-7-one, 5-[2-iso-butoxy-5-(4-ethylpiperazin-1-ylsulfonyl)pyridin-3-yl]-3-ethyl-2-(1-methylpiperidin-4-yl)-2,6-dihydro-7H-pyrazolo[4,3-d]pyrimidin-7-one, 5-[2-ethoxy-5-(4-ethylpiperazin-1-ylsulfonyl)pyridin-3-yl]-3-ethyl-2-phenyl-2,6-dihydro-7H-pyrazolo[4,3-d]pyrimidin-7-one, 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one, 4-(4-chlorobenzyl)amino-6,7,8-trimethoxyquinazoline, 7,8-dihydro-8-oxo-6-[2-propoxyphenyl]-1H-imidazo[4,5-g]quinazoline, 1-[3-[1-[(4-fluorophenyl)methyl]-7,8-dihydro-8-oxo-1H-imidazo[4,5-g]quinazolin-6-yl]-4-propoxyphenyl]carboxamide, 2-[2-ethoxy-5-(4-ethyl-piperazine-1-sulfonyl)-phenyl]-5-methyl-7-propyl-3H-imidazo[5,1-f]-[1,2,4]-triazin-4-one, and 1-{6-ethoxy-5-[3-ethyl-6,7-dihydro-2-(2-methoxyethyl)-7-oxo-2H-pyrazolo[4,3-d]pyrimidin-5-yl]-3-pyridylsulfonyl}-4-ethylpiperazine, optionally in form of the pharmaceutically acceptable acid addition salts, in form of the hydrates and/or solvates and optionally in the form of the individual optical isomers, mixtures of the individual enantiomers or racemates thereof.

Particularly preferred within the compositions according to the invention are pharmaceutical compositions comprising a therapeutically effective amount of flibanserin 1 and a therapeutically effective amount of one or more, preferably one compound 2c selected from among vardenafil, sildenafil, tadalafil, NCX-911, Sch-444877, FR-229934, 4-bromo-5-(pyridylmethylamino)-6-[3-(4-chlorophnyl)-propoxy]-3(2H)pyridazinone, 1-[4-(1,3-benzodioxol-5-ylmethyl)amiono]-6-chloro-2-[quinozolinyl]-4-piperidine-carboxylic acid, monosodium salt, (+)-cis-5,6a,7,9,9,9a- hexahydro-2-[4-(trifluoromethyl)-phenylmethyl-5-methyl-cyclopent4,5]imidazo[2,1-b]purin-4(3H)one, furaziocillin, cis-2-hexyl-5-methyl-3,4,5,6a,7,8,9,9a-octahydrocyclopent[4,5]-imidazo[2,1-b]purin-4-one, 3-acetyl-1-(2-chlorobenzyl)-2 propylindole-6-carboxylate, 3-acetyl-1-(2-chlorobenzyl)-2-propylindole-6-carboxylate, 4-bromo-5-(3-pyridylmethylamino)-6-(3-(4-chlorophenyl) propoxy)-3-(2H)pyridazinone, 1-methyl-5(5-morpholinoacetyl-2-n-propoxyphenyl)-3-n-propyl-1,6-dihydro-7H-pyrazolo(4,3-d)pyrimidin-7-one, 1-[4-[(1,3-benzodioxol-5-ylmethyl)amino]-6-chloro-2-quinazolinyl}-4-piperidinecarboxylic acid, monosodium salt, GF-1 96960, E-8010, E-4010, Bay-38-3045, Bay-38-9456, FR226807, Sch-51866, 5-[2-ethoxy-5-(4-ethylpiperazin-1-ylsulfonyl)pyridin-3-yl]-3-ethyl-2-[2-methoxyethyl]-2,6-dihydro-7H-pyrazolo[4,3-d]pyrimidin-7-one, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one, 2-[2-ethoxy-5-(4-ethyl-piperazine-1-sulfonyl)-phenyl]-5-methyl-7-propyl-3H-imidazo[5,1-f]-[1,2,4]-triazin4-one, and 1-{6-ethoxy-5-[3-ethyl-6,7-dihydro-2-(2-methoxyethyl)-7-oxo-2H-pyrazolo[4,3-d]pyrimidin-5-yl]-3-pyridylsulfonyl}-4-ethylpiperazine, optionally in form of the pharmaceutically acceptable acid addition salts, in form of the hydrates and/or solvates and optionally in the form of the individual optical isomers, mixtures of the individual enantiomers or racemates thereof.

Particularly even more preferred within the compositions according to the invention are pharmaceutical compositions comprising a therapeutically effective amount of flibanserin 1 and a therapeutically effective amount of one or more, preferably one compound 2c selected from the group consisting of vardenafil, sildenafil, tadalafil, 5-[2-ethoxy-5-(4-ethylpiperazin-1-ylsulfonyl)pyridin-3-yl]-3-ethyl-2-[2-methoxyethyl]-2,6-dihydro-7H-pyrazolo[4,3-d]pyrimidin-7-one, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one, 2-[2-ethoxy-5-(4-ethyl-piperazine-1-sulfonyl)-phenyl]-5-methyl-7-propyl-3H-imidazo[5,1-f]-[1,2,4]-triazin-4-one, and 1-{6-ethoxy-5-[3-ethyl-6,7-dihydro-2-(2-methoxyethyl)-7-oxo-2H-pyrazolo[4,3-d]pyrimidin-5-yl]-3-pyridylsulfonyl}-4-ethylpiperazine, with vardenafil, sildenafil and tadalafil being particular preferred, optionally in form of the pharmaceutically acceptable acid addition salts, in form of the hydrates and/or solvates and optionally in the form of the individual optical isomers, mixtures of the individual enantiomers or racemates thereof.

Another preferred set of compounds 2c applicable within the scope of the instant invention are compounds of formula 2c.1 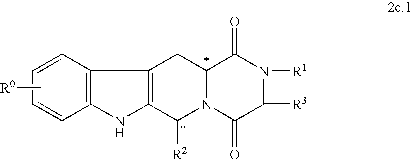
wherein
R⁰ represents hydrogen, halogen or C₁₋₆alkyl;
R¹ represents hydrogen, C₁₋₆alkyl, C₂₋₆alkenyl, C₂₋₆alkynyl, haloC₁₋₆alkyl, C₃₋₈cycloalkyl, C₃₋₈cycloalkyl-C₁₋₃alkyl, arylC₁₋₃alkyl or heteroaryl_{C1-3}alkyl;
R² represents an optionally substituted monocyclic aromatic ring selected from benzene, thiophene, furan and pyridine or an optionally substituted bicyclic ring 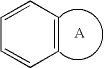
   attached to the rest of the molecule via one of the benzene ring carbon atoms and wherein the fused ring A is a 5- or 6-membered ring which may be saturated or partially or fully unsaturated and comprises carbon atoms and optionally one or two heteroatoms selected from oxygen, sulphur and nitrogen; and
R³ represents hydrogen or C₁₋₃alkyl, or R¹ and R³ together represent a 3- or 4-membered alkyl or alkenyl chain, optionally in form of the pharmaceutically acceptable acid addition salts, in form of the hydrates and/or solvates and optionally in the form of the individual optical isomers, mixtures of the individual enantiomers or racemates thereof.

The aforementioned compounds of formula 2c.1 are known in the art (WO 95/19978).

Another preferred genus of compounds 2c applicable within the scope of the instant invention are compounds of formula 2c.2 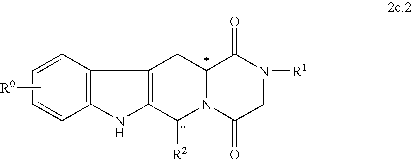
wherein
R⁰ represents hydrogen, halogen or C₁₋₆alkyl;
R¹ represents hydrogen,C₁₋₆alkyl, haloC₁₋₆alkyl, C₃₋₈cycloalkyl, C₃₋₈cycloalkyC₁₋₃alkyl, arylC₁₋₃alkyl or heteroarylC₁₋₃alkyl; and
R² represents an optionally substituted monocyclic aromatic ring selected from benzene, thiophene, furan and pyridine or an optionally substituted bicyclic ring 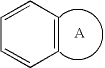
   attached to the rest of the molecule via one of the benene ring carbon atoms and wherein the fused ring A is a 5- or 6-membered ring which may be saturated or partially or fully unsaturated and comprises carbon atoms and optionally one or two heteroatoms selected from oxygen, sulphur and nitrogen, optionally in form of the pharmaceutically acceptable acid addition salts, in form of the hydrates and/or solvates and optionally in the form of the individual optical isomers, mixtures of the individual enantiomers or racemates thereof.

The aforementioned compounds of formula 2c.2 are known in the art (WO 95/19978).

Another preferred embodiment according to the invention is directed to pharmaceutical compositions comprising a therapeutically effective amount of flibanserin 1 and a therapeutically effective amount of one or more, preferably one 5-HT-1A agonist 2d, optionally in combination with a pharmaceutically acceptable excipient. Examples of suitable 5-HT-1A agonists include Urapidil, Buspirone, Aripiprazole, Ziprasidone, Naftopidil, Tandospirone, Nemonapride, Gepirone, Repinotan, Sumanirole, Xaliproden hydrochloride, Bifeprunox, AP-521, SUN-N4057, Sarizotan, MKC-242, OPC-14523, Eptapirone maleate, SLV-308, BTS-79018, R-137696, F-13640, SSR-181507, SLV-314, SLV-319, 7-OH-DPAT, VN-2222, PD-158771, RS-30199, WAY-100012, A-74283, Enilospirone, Org-13011, B-8805-033, AP-159, AZ-16596, Anpirtoline, Ebalzotan, Binospirone, MDL-72832, RU-24969, Bay-r-1531, Ipsapirone, BIMG 80, BMS-181100, BMS-181101, BMS-181970, BMY-7378, BW-1205U90, B-20991, HAT-90B, Nerisopam, LY-175644, LY-178210, LY-228729, LY-274600, LY-274601, LY-293284, LY-301317, LY-315535, E-4414, E-6265 citrate, Lesopitron, RGH-1756, RGH-1757, 1192U90, HP-236, FG-5938, LEK-8804, LB-50016, RWJ-25730, EMD-56551, EMD-67478, EMD-77697, Roxindole, Vilazodone, BP-554, CGP-50281, CGS-12066B, CGS-18102, SDZ-MAR-327, CL-870801, CP-110330, CP-146662, CP-291952, FCE-23892, FG-5865, FG-5893, OSU-191, Sunepitron, U-67413B, U-86170, U-86192A, U-92016A, U-93385, Eptapirone, Mazapertine succinate, SL-870765, SL-880338, SR-59026, Bromerguride, Alnespirone, S-14506, S-14671, S-15535, S-15931, S-16924, S-213571, S-215521, Elopiprazole, Eltoprazine, Flesinoxan, Umespirone, SUN-8399, S-23751, PM-1000, LY 41, Adatanserin, WY-48723, Zalospirone and MDL-73975, optionally in form of the pharmaceutically acceptable acid addition salts, in form of the hydrates and/or solvates and optionally in the form of the individual optical isomers, mixtures of the individual enantiomers or racemates thereof.

Preferred examples of suitable 5-HT-1A agonists 2d include Urapidil, Buspirone, Aripiprazole, Ziprasidone, Naftopidil, Tandospirone, Nemonapride, Gepirone, Repinotan, Sumanirole, Xaliproden hydrochloride, Bifeprunox, AP-521, SUN-N4057, Sarizotan, MKC-242, OPC-14523, Eptapirone maleate, SLV-308, BTS-79018, R-137696, F-13640, SSR-181507, SLV-314, SLV-319, 7-OH-DPAT, VN-2222, PD-158771, RS-30199 and WAY-100012, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one dopamine agonist 2e, optionally in combination with a pharmaceutically acceptable excipient. Examples of suitable dopamine agonists 2e include ABT-724, CP-226269, bromocriptin, cabergolin, alpha-dihydroergocryptin, lisuride, pergolide, pramipexol, roxindol, ropinirol, sopirinol, talipexol, bupropion and terguride optionally in form of the pharmaceutically acceptable acid addition salts, in form of the hydrates and/or solvates and optionally in the form of the individual optical isomers, mixtures of the individual enantiomers or racemates thereof.

Preferred examples of suitable dopamine agonist 2e include pramipexol, bupropion roxindol, and talipexol, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one 5-HT2A/2C antagonist 2f, optionally in combination with a pharmaceutically acceptable excipient. Examples of suitable 5-HT2A/2C antagonists 2f include Aripiprazole, Fluoxetine, Nefazodone, Pizotifen, Risperidone, Sarpogrelate, Ziprasidone, Agomelatine, Asenapine, Eplivanserin, lloperidone, ketanserin, ritanserin, M 100907, Netamiftide, Ocaperidone, S-20098, Abaperidone, ACP-103, EMD 281014, EMR 62218, LU-31-130, SL 650472, EGIS-10037, LEK-8829, Nantenine, QF-2004B, R-107500, S 35120, S-14297, Amesergide, Amperozide, AT 1015, Balaperidone, BIMG 80, Deramciclane, EGIS 8465, EGIS 9933, Fananserin, FG 5803, FG 5893, FG-5938, FG-5974, GMC 1169, GMC 283, GMC 306, GMC 6139, ICI-169369, Irindalone, IT 657, JL-13, Lubazodone, LY 215840, LY-367265, NRA-0045, Org-38457, PNU-96415E, QF 0510B, QF 1003B, QF 1004B, RO 600946, Ro-60-0759, RP 71602, RS-102221, S 16924, S 213571, S 35031, S-1 7828, S-21357-1, SB 200646A, SB 206553, SB 221284, SB 228357, SB 242084, SB 243213, SDZ SER 082, TY 12283, TY-11223 and ZD-3638 optionally in form of the pharmaceutically acceptable acid addition salts, in form of the hydrates and/or solvates and optionally in the form of the individual optical isomers, mixtures of the individual enantiomers or racemates thereof.

Preferred 5-HT2A/2C antagonist 2f include Aripiprazole, Fluoxetine, Nefazodone, Pizotifen, Risperidone, Sarpogrelate, Ziprasidone, Agomelatine, Asenapine, Eplivanserin, Iloperidone, M 100907, Netamiftide, Ocaperidone, S-20098, Abaperidone, ketanserin, ritanserin, ACP-103, EMD 281014, EMR 62218, LU-31-130, SL 650472, EGIS-10037, LEK-8829, Nantenine, QF-2004B, R-107500, S 35120 and S-14297optionally in form of the pharmaceutically acceptable acid addition salts, in form of the hydrates and/or solvates and optionally in the form of the individual optical isomers, mixtures of the individual enantiomers or racemates thereof. Particular preferred 5-HT2A/2C antagonist 2f are selected from the group consisting of Aripiprazole, Fluoxetine, Nefazodone, Pizotifen, Risperidone, Sarpogrelate and Ziprasidone optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one dopamine D4 antagonist 2g, optionally in combination with a pharmaceutically acceptable excipient. Examples of suitable dopamine D4 antagonists 2g include olanzapine, ziprasidone, MDL-814608A, NRA-0562, S-18126, SPI-376, YM-50001, 1192U90, ALX-D4, Balaperidone, BIMG 80, CI-1030, CP-293019, Fananserin, JL-13, L-741742, L-745870, L-751852, L-772620, L-800892, LU-35138, LUR-2366, NEO-376, NGB-4420, NGD-941, NRA-0045, NRA-0074, NRA-0154, NRA-0160, NRA-0161, NRA-0215, NRA-0219, NRA-0544, PD-089232, PD-108306, PD-165167, PD-167063, PD-168306, PD-172760, PD-172760, PD-172938, PD-35680, PD-82011, PNU-106161, PNU-106675, QF-1003B, QF-1004B, Ro-62-4599, S-16924, S-17828, Sch-71450, Sonepiprazole, U-101958, U-103073E, U-96415E and YM43611, optionally in form of the pharmaceutically acceptable acid addition salts, in form of the hydrates and/or solvates and optionally in the form of the individual optical isomers, mixtures of the individual enantiomers or racemates thereof.

Preferred examples of suitable dopamine D4 antagonist 2g include olanzapine, ziprasidone, MDL-814608A, NRA-0562, S-18126, SPI-376 and YM-50001, in particular olanzapine and ziprasidone, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one selective androgen receptor modulator (SARM) 2h, optionally in combination with a pharmaceutically acceptable excipient. Examples of suitable SARMs 2h include LGD2226, LGD1 331, (both available from Ligand Pharmaceuticals (San Diego, Calif.)), bicalutamide, cyproterone acetate, hydroxyflutamide, spironolactone, 4-(trifluoromethyl)-2(1 H)-pyrrolidone[3,2-g]quinolinone and its derivatives, 1,2-dihydropyridono[5,6-g]quinoline and its derivatives and piperidino[3,2-g]quinolinone and its derivatives, optionally in form of the pharmaceutically acceptable acid addition salts, in form of the hydrates and/or solvates and optionally in the form of the individual optical isomers, mixtures of the individual enantiomers or racemates thereof.

Preferred examples of suitable SARMs 2h include LGD2226 and/or LGD1 331, bicalutamide, cyproterone acetate, hydroxyflutamide and spironolactone, in particular LGD2226, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one estrogen 2k, optionally in combination with a pharmaceutically acceptable excipient. Examples of suitable estrogens 2k include synthetic and natural estrogens such as estradiol (i.e. 1,3,5-estratriene-3,17β-diol, or “17β-estradiol”) and its esters, including estradiol benzoate, valerate, cypionate, heptanoate, decanoate, acetate and diacetate, 17α-estradiol, ethinylestradiol (i.e. 17α-ethynylestradiol) and esters and ethers thereof, including ethinylestradiol 3-acetate and ethinylestradiol 3-benzoate, estriol and estriol succinate, polyestrol phosphate, estrone and its esters and derivatives, including estrone acetate, estrone sulfate, and piperazine estrone sulfate, quinestrol, mestranol, and conjugated equine estrogens, optionally in form of the pharmaceutically acceptable acid addition salts, in form of the hydrates and/or solvates and optionally in the form of the individual optical isomers, mixtures of the individual enantiomers or racemates thereof.

Preferred examples of suitable estrogens 2k include estradiol and 17α-estradiol, in particular estradiol, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one androgen 2l, optionally in combination with a pharmaceutically acceptable excipient. Examples of suitable androgens 2l include, but are not limited to the naturally occurring androgens and derivatives thereof, including androsterone, androsterone acetate, androsterone propionate, androsterone benzoate, androstenediol, androstenediol-3-acetate, androstenediol-17-acetate, androstenediol-3,17-diacetate, androstenediol-1 7-benzoate, androstenediol-3-acetate-1 7-benzoate, androstenedione, ethylestrenol, oxandrolone, nandrolone phenpropionate, nandrolone decanoate, nandrolone furylpropionate, nandrolone cyclohexane-propionate, nandrolone benzoate, nandrolone cyclohexanecarboxylate, stanozolol, dromostanolone, dromostanolone propionate, testosterone, dehydroepiandrosterone (“prasterone”), sodium dehydroepiandrosterone sulfate, and 4-dihydrotestosterone (“stanolone” and 5α-dihydrotestosterone); pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, pentadecanoate, undecanoate, pelargonate, tridecanoate, palmitate, caprate, isocaprate, α-methylcaprate, β-methylcaprate, laurate, α-methylpelargonate, β-methylpelargonate, ββ-dimethylpelargonate, β-(p-methyl-cyclohexyl)propionate, β-(p-ethylcyclohexyl)-propionate, β-(cycloheptyl)-propionate, α-methyl-cyclohexyl-propionate, β-methyl-β-cyclohexyl-propionate, cyclododecyl-carboxylate, adamantine-1′-carboxylate, adamant-1′-yl-acetate, methyl-α-cyclohexyl propionate, and α-(bicyclo-[2,2,2-oct-1′-yl)-propionate esters, as well as the alkyl-substituted, preferably C₄-C₆ alkyl-substituted cyclic esters, such as the 3-n-hexylcyclobutanecarboxylate, 3-n-butylcyclopentanecarboxylate, 4-n-butylcyclohexanecarboxylate, 4-n-pentylcyclohexanecarboxylate and n-hexylcyclohexanecarboxylate esters; and pharmaceutically acceptable derivatives of testosterone such as methyl testosterone, testolactone, oxymetholone, fluoxymesterone, optionally in form of the pharmaceutically acceptable acid addition salts, in form of the hydrates and/or solvates and optionally in the form of the individual optical isomers, mixtures of the individual enantiomers or racemates thereof.

Preferred examples of suitable androgens 2l include testosterone, methyl testosterone, testolactone, oxymetholone, fluoxymesterone, in particular testosterone, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one α-adrenergic receptor antagonist 2m, optionally in combination with a pharmaceutically acceptable excipient. Examples of suitable α-adrenergic receptor antagonists 2m include phentolamine mesylate, HMP-12, REC-15/2615 and MPV 1248 (atipamezole), optionally in form of the pharmaceutically acceptable acid addition salts, in form of the hydrates and/or solvates and optionally in the form of the individual optical isomers, mixtures of the individual enantiomers or racemates thereof.

Preferred examples of suitable α-adrenergic receptor antagonists 2m include phentolamine mesylate and REC-15/2615, optionally in form of the pharmaceutically acceptable acid addition salts, in form of the hydrates and/or solvates and optionally in the form of the individual optical isomers, mixtures of the individual enantiomers or racemates thereof.

Another preferred embodiment according to the invention is directed to pharmaceutical compositions comprising a therapeutically effective amount of flibanserin 1 and a therapeutically effective amount of one or more, preferably one selective estrogen receptor modulator (SERM) 2n, optionally in combination with a pharmaceutically acceptable excipient. Examples of suitable SERMs 2n include tibolone, diethylstilbestrol, moxestrol, N-butyl-3,17-dihydroxy-N-methyl-estra-1,3,5(10)-triene-7-undecanamide (ICI 164,384), fulvestrant (ICI 182,780), 19-nor-progesterone and its derivatives, and 19-nor-testosterone and its derivatives, raloxifene ([6-hydroxy-3-[4-[2-(1-piperidinyl)ethoxy]phenoxy]-2-(4-hydroxyphenyl)]benzo[b]thiophene hydrochloride), and derivatives thereof, including —S—, —NH—, —NCH₃—, —SO₂— and —CH₂—substituted raloxifene, as described in Schmid et al. ((1999) Bioorg. & Med. Chem. Lett. 9:523-528), trans-2,3-dihydroraloxifene and its derivatives as disclosed in Grese, et al., (J. Med. Chem. (1997) Vol. 40, pp.146-167) such as 4′ halo-raloxifene and 2-(alkyl, cycloalkyl or naphthyl) raloxifene, benzothiophenes as disclosed in U.S. Pat. No. 5,962,475, such as 6-methoxy-2-(4-methoxyphenyl)-3-(4-nitrobenzoyl)-benzo[b]thiophene, arzoxifene (LY353381), 2-(4-methoxyphenyl)-3-(4-(2-(1-piperidinyl)ethoxy)-phenoxybenzo(b)thiophene-6-ol); LY 117018 (6-hydroxy-2-(4-hydroxyphenyl)benzo(b)thien-3-yl)(4-(2-(1-pyrrolidinyl)ethoxy)phenyl)-methanone), and bazedoxifen (TSE-424), idoxifene (1-[2-[4-(1 E)-1-(4-lodophenyl)-2-phenyl-1-butenyl]phenoxy]ethyl]pyrrolidine) droloxifene (3-[(1E)-1-[4-[2-(Dimethylamino)ethoxy]phenyl]-2-phenyl-1-butenyl]phenol), tamoxifen ((Z)-2-[4-(1,2-Diphenyl-1-butenyl)phenoxy]-N,N-dimethylethanamine), toremifene (2-[4-[(1Z)-4-Chloro-1,2-diphenyl-1-butenyl)phenoxy]-N,N-dimethylethanamine), clomiphene, (2-[4-(2-Chloro-1,2-diphenylethenyl)phenoxy]-N,N-diethylethanamine), meproxifene ((4-(1-(4-(2-(dimethylamino)ethoxy)phenyl)-2-(4-(1-methylethyl)phenyl)-1-butenyl)-phenol) or TAT-59), trioxifene, zindoxifene, lasofoxifene, nafoxidine, halogenated triphenylethylene derivatives as disclosed in U.S. Patent Application Publication No. 2002/0013297, such as 3-[4-[1-(4-fluorophenyl)-2-phenyl-but-1-enyl]phenyl}acrylic acid and 3-[4-(1,2-diphenyl-but-1-enyl)-phenyl]-acrylic acid; substituted naphthalenes and isoquinolines, including, for example cis-6-phenyl-5-(4-(2-pyrrolidin-1-yl-ethoxy)phenyl)-5,6,7,8-tetrahydronaphthalene-2-ol, cis-6-(4-fluorophenyl)-5-[4-(2-piperidin-1-yl-ethoxy)-phenyl]-5,6,7,8-tetrahydronaphthalene-2-ol, cis-1-[6′-pyrrolidinoethoxy-3′-pyridyl]-2-phenyl-6-hydroxy-1,2,3,4-tetrahydronaphthalene, cis-6-(4′-hydroxyphenyl)-5-[4-(2-piperidin-1-yl-ethoxy)-phenyl]-5,6,7,8-tetrahydronaphthalen-2-ol, 6-(4-hydroxyphenyl)-5-[4-(2-piperidin-1-yl-ethoxy)-benzyl]-naphthalen-2-ol, 1-(4′-pyrrolidinoethoxyphenyl)-2-(4″-fluorophenyl)-6-hydroxy-1,2,3,4-tetrahydroisoquinoline, 1-(4′-pyrrolidinoethoxyphenyl)-2-phenyl-6-hydroxy-1,2,3,4 tetrahydroisoquinoline, and other compounds disclosed in U.S. Pat. No. 5,916,916, U.S. Pat. No. 5,552,412 and in EP 1004306 A2, optionally in form of the pharmaceutically acceptable acid addition salts, in form of the hydrates and/or solvates and optionally in the form of the individual optical isomers, mixtures of the individual enantiomers or racemates thereof.

Preferred examples of a suitable SERMs 2n are tibolone and lasofoxifene, optionally in form of the pharmaceutically acceptable acid addition salt, in form of the hydrate and/or solvate and optionally in the form of the individual optical isomers, mixtures of the individual enantiomers or racemates thereof.

In the present invention the term “modulator” as used in the terms “selective androgen receptor modulator” or “selective estrogen receptor modulator” means a compound that produces tissue specific effects that can be agonistic or antagonistic to the effects of estrogen or androgen.

Flibanserin 1 may be used in form of the free base, optionally in form of its pharmaceutically acceptable acid addition salts and/or optionally in form of the hydrates and/or solvates thereof. Suitable acid addition salts include for example those of the acids selected from, succinic acid, hydrobromic acid, acetic acid, fumaric acid, maleic acid, methanesulphonic acid, lactic acid, phosphoric acid, hydrochloric acid, sulphuric acid, tartaric acid and citric acid. Mixtures of the abovementioned acid addition salts may also be used. From the aforementioned acid addition salts the hydrochloride and the hydrobromide, particularly the hydrochloride, are preferred. If flibanserin 1 is used in form of the free base, it is preferably used in form of flibanserin polymorph A as disclosed in WO 03/014079.

The active ingredients 2 which are suitable to be combined with flibanserin within the teaching of the instant invention and which are mentioned hereinbefore may also be capable of forming acid addition salts with pharmaceutically acceptable acids. Representative salts include the following: Acetate, Benzenesulfonate, Benzoate, Bicarbonate, Bisulfate, Bitartrate, Borate, Bromide, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate,Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Sulfate, Subacetate, Succinate, Tannate, Tartrate, Teoclate, Tosylate, Triethiodide and Valerate.

Furthermore, where the compounds 2 carry an acidic moiety, suitable pharmaceutically acceptable salts thereof may include alkali metal salts, e.g., sodium or potassium salts; alkaline earth metal salts, e. g., calcium or magnesium salts; and salts formed with suitable organic ligands, e.g., quaternary ammonium salts.

The compounds 2 may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention. Further included are all mixtures of the two enantiomers. Also included within the scope of the invention are polymorphs and hydrates of the compounds of the instant invention.

The present invention includes within its scope prodrugs of the compounds 1 and 2. In general, such prodrugs will be functional derivatives of the compounds of this invention which are readily convertible in vivo into the required compound.

The term “therapeutically effective amount” shall mean that amount of a drug or pharmaceutical agent that will elicit the biological or medical response of a tissue, system, animal or human that is being sought by a researcher or clinician.

As used herein, the term “composition” is intended to encompass a product comprising the specified ingredients in the specified amounts, as well as any product which results, directly or indirectly, from combination of the specified ingredients in the specified amounts.

In the combination of the present invention, the components 1 and 2 may be administered separately or together in one pharmaceutical composition. In addition, the administration of one element of the combination of the present invention may be prior to, concurrent to, or subsequent to the administration of the other element of the combination.

The elements of the combination of 1 and 2 may be administered by oral, parenteral (e.g., intramuscular, intraperitoneal, intravenous or subcutaneous injection, or implant), buccal, nasal, vaginal, rectal, sublingual, or topical (e.g. ocular eyedrop) routes of administration and may be formulated, alone or together, in suitable dosage unit formulations containing conventional non-toxic pharmaceutically acceptable carriers, adjuvants and vehicles appropriate for each route of administration.

The pharmaceutical compositions for the administration of the components 1 and 2 of this invention may conveniently be presented in dosage unit form and may be prepared by any of the methods well known in the art of pharmacy. All methods include the step of bringing the active ingredient into association with the carrier which is constituted of one or more accessory ingredients. In general, the pharmaceutical compositions are prepared by uniformly and intimately bringing the active ingredients into association with a liquid carrier or a finely divided solid carrier or both, and then, if necessary, shaping the product into the desired dosage form. In the pharmaceutical compositions the active compounds are included in an amount sufficient to produce the desired pharmacologic effect.

The pharmaceutical compositions containing the active ingredients 1 and 2, separately or together, that are suitable for oral administration may be in the form of discrete units such as hard or soft capsules, tablets, troches or lozenges, each containing a predetermined amount of the active ingredients; in the form of a dispersible powder or granules; in the form of a solution or a suspension in an aqueous liquid or non-aqueous liquid; in the form of syrups or elixirs; or in the form of an oil-in-water emulsion or a water-in-oil emulsion.

Dosage forms intended for oral use may be prepared according to any method known in the art for the manufacture of pharmaceutical formulations and such compositions.

The excipients used may be for example, (a) inert diluents such as mannitol, sorbitol, calcium carbonate, pregelatinized starch, lactose, calcium phosphate or sodium phosphate; (b) granulating and disintegrating agents, such as povidone, copovidone, hydroxypropylmethylcellulose, corn starch, alginic acid, crospovidone, sodiumstarchglycolate, croscarmellose, or polacrilin potassium; (c) binding agents such as microcrystalline cellulose or acacia; and (d) lubricating agents such as magnesium stearate, stearic acid, fumaric acid or talc.

In some cases, formulations for oral use may be in the form of hard gelatin or HPMC capsules wherein the active ingredient 1 or 2, separately or together, is mixed with an inert solid diluent, for example pregelatinized starch, calcium carbonate, calcium phosphate or kaolin, or dispensed via a pellet formulation. They may also be in the form of soft gelatin capsules wherein the active ingredient is mixed with water or an oil medium, for example peanut oil, liquid paraffin, medium chain triglycerides or olive oil.

The tablets, capsules or pellets may be uncoated or they may be coated by known techniques to delay disintegration and absorption in the gastrointestinal tract and thereby provide a delayed action or sustained action over a longer period. For example, a time delay material such as celluloseacetate phtalate or hydroxypropylcellulose acetate succinate or sustained release material such as ethylcellulose or ammoniomethacrylate copolymer (type B) may be employed.

Liquid dosage forms for oral administration include pharmaceutically acceptable emulsions, solutions, suspensions, syrups, and elixirs containing inert diluents commonly used in the art, such as water. Besides such inert diluents, compositions can also include adjuvants, such as wetting agents, emulsifying and suspending agents, and sweetening, flavoring, perfuming and preserving agents.

Aqueous suspensions normally contain the active materials 1 and 2, separately or together, in admixture with excipients suitable for the manufacture of aqueous suspensions. Such excipients may be (a) suspending agents such as hydroxy ethylcellulose, sodium carboxymethylcellulose, methylcellulose, hydroxypropylmethylcellulose, sodium alginate, polyvinylpyrrolidone, gum tragacanth and gum acacia; (b) dispersing or wetting agents which may be (b.1) a naturally-occurring phosphatide such as lecithin, (b.2) a condensation product of an alkylene oxide with a fatty acid, for example, polyoxyethylene stearate, (b.3) a condensation product of ethylene oxide with a long chain aliphatic alcohol, for example heptadecaethyleneoxycetanol, (b.4) a condensation product of ethylene oxide with a partial ester derived from a fatty acid and a hexitol such as polyoxyethylene sorbitol monooleate, or (b.5) a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents, such as sucrose or saccharin.

Oily suspensions may be formulated by suspending the active ingredients 1 and 2, separately or together, in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be prepared by the addition of an antioxidant such as ascorbic acid.

Dispersible powders and granules are suitable for the preparation of an aqueous suspension. They provide the active ingredients 1 and 2, separately or together, in admixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

The pharmaceutical compositions of the invention may also be in the form of oil-in-water emulsions. The oily phase may be a vegetable oil such as olive oil or arachis oils, or a mineral oil such as liquid paraffin or a mixture thereof.

Suitable emulsifying agents may be (a) naturally-occurring gums such as gum acacia and gum tragacanth, (b) naturally-occurring phosphatides such as soybean and lecithin, (c) esters or partial esters derived from fatty acids and hexitol anhydrides, for example, sorbitan monooleate, (d) condensation products of said partial esters with ethylene oxide, for example polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a preservative and flavoring and coloring agents.

The pharmaceutical compositions containing 1 and 2, separately or together, may be in the form of a sterile injectable aqueous or oleagenous suspension or solution. The suspension may be formulated according to known methods using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane-diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

Preparations according to this invention containing 1 and 2, separately or together, for parenteral administration include sterile aqueous or non-aqueous solutions, suspension, or emulsions.

Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be reconstituted in sterile water, or some other sterile injectable medium immediately before use. The combination of this invention may also be administered in the form of suppositories for rectal administration. This composition can be prepared by mixing the drugs with a suitable non-irritating excipient which is solid at ordinary temperatures, e.g., room temperature, but liquid at the rectal temperature and will therefore melt in the rectum to release the drug. Such materials are cocoa butter, hard fat, and polyethylene glycols. Compositions for buccal, nasal or sublingual administration are also prepared with standard excipients well known in the art.

For topical administration the combinations of this invention containing 1 and 2, separately or together, may be formulated in liquid or semi-liquid preparations such as liniments, lotions, applications; oil-in-water or water-in-oil emulsions such as creams, ointments, jellies or pastes, including tooth-pastes; or solutions or suspensions such as drops, and the like.

The dosage of the active ingredients in the compositions of this invention may be varied. However, it is necessary that the amount of the active ingredients 1 and 2 be such that a suitable dosage form is obtained. The selected dosage and the dosage form depend upon the desired therapeutic effect, on the route of administration and on the duration of the treatment. Dosage ranges in the combination are approximately one tenth to one times the clinically effective ranges required to induce the desired therapeutic effect, respectively when the compounds are used singly.

Within the instant invention flibanserin 1 is preferably administered in such an amount that per single dosage between 5 to 200 mg of flibanserin 1 are applied. Preferred are ranges of between 10 to 150 mg, particular preferred 20 to 100 mg of flibanserin 1. Suitable dosage forms may contain for instance 20, 25, 30, 35, 40, 45, 50, 55, 60, 65, 70, 75, 80, 85, 90, 95 or 100 mg of flibanserin 1. The aforementioned values are based on flibanserin 1 in form of the free base. If flibanserin 1 is applied in form of one of its acid addition salts, the corresponding values are readily calculable from the aforementioned values.

Within the instant invention the melanocortin agonist 2a is preferably administered in a range of between about 0.001 mg per kg of bodyweight per day (mg/kg/day) to about 100 mg/kg/day, preferably 0.01 to 10 mg/kg/day, and most preferably 0.1 to 5.0 mg/kg/day. Intravenously, the most preferred doses will range from about 0.1 to about 10 mg/kg/minute during a constant rate infusion. Advantageously, the compounds 2a of the present invention may be administered in a single daily dose, or the total daily dosage may be administered in divided doses of two, three or four times daily.

Within the instant invention the prostaglandin E1 agonist 2b is preferably administered in such an amount that per day between 0.1 to 150 μg are applied. Preferred are ranges of between 0.5 to 100 μg, particular preferred 1 to 50 μg of the prostaglandin E1 agonist 2b. In case of the preferred prostaglandin E1 agonist 2b limaprost particularly preferred doses per day are in the range of about 15 to 30 μg. In case of the preferred prostaglandin E1 agonist 2b alprostadil particularly preferred doses per day are in the range of about 1.25 to 20 μg. Suitable dosage forms may contain for instance 1, 5, 10, 15, 20, 25, 30, 35, 40, 45 or 50 μg of the prostaglandin E1 agonist 2b. Advantageously, the compounds 2b of the present invention may be administered in a single daily dose, or the total daily dosage may be administered in divided doses of two, three or four times daily.

Within the instant invention the elevator of cGMP 2c is preferably administered in such an amount that per day between 0.1 to 200 mg of 2c are applied. Preferred are ranges of between 1 to 150 mg, particular preferred 5 to 100 mg of 2c. In case of the preferred elevator of cGMP 2c sildenafil particularly preferred doses per day are in the range of about 25 to 100 mg. In case of the preferred elevator of cGMP 2c tadalafil particularly preferred doses per day are in the range of about 10 to 20 mg. In case of the preferred elevator of cGMP 2c vardenafil particularly preferred doses per day are in the range of about 5 to 20 mg. Suitable dosage forms may contain for instance 5, 10, 15, 20, 25, 30, 35, 40, 45, 50, 55, 60, 65, 70, 75, 80, 85, 90, 95 or 100 mg of 2c. Advantageously, the compounds 2c of the present invention may be administered in a single daily dose, or the total daily dosage may be administered in divided doses of two, three or four times daily.

Within the instant invention the 5-HT-1A agonist 2d is preferably administered in such an amount that per day between 1 to 200 mg of 2d are applied. Preferred are ranges of between 5 to 150 mg, particular preferred 10 to 100 mg of 2d. In case of the preferred 5-HT-1A agonist 2d aripiprazole particularly preferred doses per day are in the range of about 10 to 30 mg. In case of the preferred 5-HT-1A agonist 2d ziprasidone particularly preferred doses per day are in the range of about 20 to 80 mg. Suitable dosage forms may contain for instance 10, 15, 20, 25, 30, 35, 40, 45, 50, 55, 60, 65, 70, 75, 80, 85, 90, 95 or 100 mg of 2d. Advantageously, the compounds 2d of the present invention may be administered in a single daily dose, or the total daily dosage may be administered in divided doses of two, three or four times daily.

Within the instant invention the dopamine agonist 2e is preferably administered in such an amount that per day between 0.01 to 600 mg of 2e are applied. Preferred are ranges of between 0.025 to 500 mg, particular preferred 0.05 to 450 mg of 2e. In case of the preferred dopamine agonist 2e pramipexole particularly preferred doses per day are in the range of about 0.375 to 4.5 mg. In case of the preferred dopamine agonist 2e ropinirole particularly preferred doses per day are in the range of about 0.75 to 3 mg. In case of the preferred dopamine agonist 2e bupropion particularly preferred doses per day are in the range of about 100 to 450 mg. In case of the preferred dopamine agonist 2e pergolide particularly preferred doses per day are in the range of about 0.05 to 3 mg. Suitable dosage forms may contain for instance 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445 or 450 mg of 2e. Advantageously, the compounds 2e of the present invention may be administered in a single daily dose, or the total daily dosage may be administered in divided doses of two, three or four times daily.

Within the instant invention the 5-HT2A/2C antagonist 2f is preferably administered in such an amount that per day between 0.1 to 200 mg of 2f are applied. Preferred are ranges of between 0.5 to 150 mg, particular preferred 1 to 100 mg of 2f. In case of the preferred 5-HT2A/2C antagonist 2f fluoxetine particularly preferred doses per day are in the range of about 20 to 60 mg. In case of the preferred 5-HT2A/2C antagonist 2f risperidone particularly preferred doses per day are in the range of about 1 to 8 mg. Suitable dosage forms may contain for instance 1, 2, 3, 4, 5, 6, 7, 8, 9, 10,15, 20, 25, 30, 35, 40, 45, 50, 55, 60, 65, 70, 75, 80, 85, 90, 95 or 100 mg of 2f. Advantageously, the compounds 2f of the present invention may be administered in a single daily dose, or the total daily dosage may be administered in divided doses of two, three or four times daily.

Within the instant invention the dopamine D4 antagonist 2g is preferably administered in such an amount that per day between 0.1 to 100 mg of 2g are applied. Preferred are ranges of between 1 to 75 mg, particular preferred 5 to 50 mg of 2g. In case of the preferred dopamine D4 antagonist 2g olanzapine particularly preferred doses per day are in the range of about 5 to 15 mg. Suitable dosage forms may contain for instance 5, 10, 15, 20, 25, 30, 35, 40, 45 or 50 mg of 2g. Advantageously, the compounds 2g of the present invention may be administered in a single daily dose, or the total daily dosage may be administered in divided doses of two, three or four times daily.

Within the instant invention the selective androgen receptor modulator (SARM) 2h is preferably administered in such an amount that per day between 0.01 to 600 mg of 2h are applied. Preferred are ranges of between 0.025 to 500 mg, particular preferred 0.05 to 100 mg of 2h.

Suitable dosage forms may contain for instance 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445 or 450 mg of 2h.

Advantageously, the compounds 2h of the present invention may be administered in a single daily dose, or the total daily dosage may be administered in divided doses of two, three or four times daily.

Within the instant invention the estrogen 2k is preferably administered in such an amount that per day between 0.1 to 3000 μg are applied. Preferred are ranges of between 0.5 to 1500 μg, particular preferred 1 to 750 μg of estrogen 2k. In case of the preferred estrogen 2k estradiol particularly preferred doses per day are in the range of about 1 μg to 500 μg, more preferrably in the range of 5 to 250 μg. Suitable dosage forms may contain for instance 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 250, 260, 270, 280, 290, 300, 310, 320, 330, 350, 375, 400, 425, 450, 475, 500, 550, 600, 650, 700, 750 μg of the estrogen 2k.

Advantageously, the compounds 2k of the present invention may be administered in a single daily dose, or the total daily dosage may be administered in divided doses of two, three or four times daily.

Within the instant invention the androgen 2l is preferably administered in such an amount that per day between 0.01 to 600 mg of 2l are applied. Preferred are ranges of between 0.025 to 500 mg, particular preferred 0.05 to 450 mg of 21. In case of the preferred androgen 2l testosterone particularly preferred doses per day are in the range of about 100 μg to 10 mg, more preferrably in the range of 500 μg to 5 mg. Suitable dosage forms may contain for instance 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445 or 450 mg of 2l.

Advantageously, the compounds 2l of the present invention may be administered in a single daily dose, or the total daily dosage may be administered in divided doses of two, three or four times daily.

Within the instant invention the α-adrenergic receptor antagonist 2m is preferably administered in such an amount that per day between 0.01 to 600 mg of 2m are applied. Preferred are ranges of between 0.025 to 500 mg, particular preferred 0.05 to 450 mg of 2m. In case of the α-adrenergic receptor antagonist 2m phentolamine mesylate preferred doses per day are in the range of about 1 to 70 mg, particularly preferred doses per day are in the range of 30 to 50 mg.

Suitable dosage forms may contain for instance 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5,10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445 or 450 mg of 2m.

Advantageously, the compounds 2m of the present invention may be administered in a single daily dose, or the total daily dosage may be administered in divided doses of two, three or four times daily.

Within the instant invention the selective androgen receptor modulator (SERM) 2n is preferably administered in such an amount that per day between 0.01 to 600 mg of 2n are applied. Preferred are ranges of between 0.025 to 500 mg, particular preferred 0.05 to 450 mg of 2n. In case of the preferred SERM 2n lasofoxifene particularly preferred doses per day are in the range of about 0.5 to 50 mg. In case of the preferred compound 2n tibolon preferred doses per day are in the range of about 0.5 to 10 mg, particularly preferred doses per day are in the range of 1 to 5 mg. Suitable dosage forms may contain for instance 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445 or 450 mg of 2n.

Advantageously, the compounds 2n of the present invention may be administered in a single daily dose, or the total daily dosage may be administered in divided doses of two, three or four times daily.

In another preferred embodiment the invention relates to a method for the treatment of disorders selected from the group consisting of Hypoactive Sexual Desire Disorder, loss of sexual desire, lack of sexual desire, decreased sexual desire, inhibited sexual desire, loss of libido, libido disturbance, and frigidity, comprising the administration of a therapeutically effective amount of 1 optionally in form of its pharmaceutically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition. In another preferred embodiment the invention relates to a method for the treatment of disorders selected from the group consisting of Hypoactive Sexual Desire Disorder, loss of sexual desire, lack of sexual desire, decreased sexual desire, inhibited sexual desire, comprising the administration of a therapeutically effective amount of 1 optionally in form of its pharmaceutically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition. In another preferred embodiment the invention relates to a method for the treatment of disorders selected from the group consisting of Hypoactive Sexual Desire Disorder and loss of sexual desire, preferably Hypoactive Sexual Desire Disorder, comprising the administration of a therapeutically effective amount of 1 optionally in form of its pharmaceutically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment of premenstrual disorders, comprising the administration of a therapeutically effective amount of 1 optionally in form of its pharmaceutically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment of premenstrual disorders, selected from the group consisting of premenstrual dysphoria, premenstrual syndrome and premenstrual dysphoric disorder, in particular premenstrual dysphoric disorder, comprising the administration of a therapeutically effective amount of 1 optionally in form of its pharmaceutically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment of sexual aversion disorder in females, comprising the administration of a therapeutically effective amount of 1 optionally in form of its pharmaceutically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment of sexual arousal disorder in females, comprising the administration of a therapeutically effective amount of 1 optionally in form of its pharmaceutically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment of orgasmic disorder in females, comprising the administration of a therapeutically effective amount of 1 optionally in form of its pharmaceutically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment of sexual pain disorders in females, comprising the administration of a therapeutically effective amount of 1 optionally in form of its pharmaceutically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In a particular preferred embodiment the invention relates to a method for the treatment of sexual pain disorders selected from the group consisting of dyspareunia, vaginismus, noncoital sexual pain disorder, sexual dysfunction due to a general medical condition and substance-induced sexual dysfunction, comprising the administration of a therapeutically effective amount of 1 optionally in form of its pharmaceutically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

The beneficial effects of the compositions according to the invention can be observed regardless of whether the disturbance existed lifelong or was acquired, and independent of etiologic origin (organic—both, physically and drug induced-, psychogen, a combination of organic—both, physically and drug induced-, and psychogen, or unknown).

Another embodiment of the invention relates to the use of the combinations of 1 and 2, optionally in form of their pharmaceutically acceptable acid addition salts for the preparation of a medicament for the treatment of the aforementioned disorders.

Another preferred embodiment of the invention is directed to the aforementioned methods wherein 2 is selected from the group consisting of the aforementioned melanocortin agonists, prostaglandin E1 agonists, elevators of cyclic guanosine 3′, 5′-monophosphate (cGMP) (preferably PDE V inhibitors), 5-HT-1A agonists, dopamine agonists, dopamine D4 antagonists, 5-HT-2A/C antagonists, selective androgen receptor modulators (SARMs), selective estrogen receptor modulators (SERMs), estrogens, androgens and α-adrenergic receptor antagonists.

Another embodiment of the invention relates to the use of the combinations of 1 and 2, optionally in form of their pharmaceutically acceptable acid addition salts for the preparation of a medicament for the treatment of the aforementioned disorders, wherein 2 is selected from the group consisting of the aforementioned melanocortin agonists, prostaglandin E1 agonists, elevators of cyclic guanosine 3′,5′-monophosphate (cGMP) (preferably PDE V inhibitors), 5-HT-1A agonists, dopamine agonists, dopamine D4 antagonists, 5-HT-2A/C antagonists, selective androgen receptor modulators (SARMs), selective estrogen receptor modulators (SERMs), estrogens, androgens and α-adrenergic receptor antagonist.

Another preferred embodiment of the invention is directed to a method for the treatment of one of the aforementioned disorders, comprising the administration of a therapeutically effective amount of one or more, preferably one of the aforementioned melanocortin agonists 2a, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of a therapeutically effective amount of one or more, preferably one of the aforementioned melanocortin agonists 2a, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment of the aforementioned disorders.

Another preferred embodiment of the invention is directed to a method for the treatment of one of the aforementioned disorders, comprising the administration of a therapeutically effective amount of one or more, preferably one of the aforementioned prostaglandin E1 agonists 2b, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of of a therapeutically effective amount of one or more, preferably one of the aforementioned prostaglandin E1 agonists 2b, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment of the aforementioned disorders.

Another preferred embodiment of the invention is directed to a method for the treatment of one of the aforementioned disorders, comprising the administration of a therapeutically effective amount of one or more, preferably one of the aforementioned elevators of cyclic guanosine 3′,5′-monophosphate (cGMP) 2c, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of a therapeutically effective amount of one or more, preferably one of the aforementioned elevators of cyclic guanosine 3′,5′-monophosphate (cGMP) 2c, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment of the aforementioned disorders.

Another preferred embodiment of the invention is directed to a method for the treatment of one of the aforementioned disorders, comprising the administration of a therapeutically effective amount of one or more, preferably one of the aforementioned 5-HT-1A agonists 2d, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of a therapeutically effective amount of one or more, preferably one of the aforementioned 5-HT-1A agonists 2d, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment of the aforementioned disorders.

Another preferred embodiment of the invention is directed to a method for the treatment of one of the aforementioned disorders, comprising the administration of a therapeutically effective amount of one or more, preferably one of the aforementioned dopamine agonists 2e, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of a therapeutically effective amount of one or more, preferably one of the aforementioned dopamine agonists 2e, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment of the aforementioned disorders.

Another preferred embodiment of the invention is directed to a method for the treatment of one of the aforementioned disorders, comprising the administration of a therapeutically effective amount of one or more, preferably one of the aforementioned 5-HT-2A/C antagonists 2f, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of a therapeutically effective amount of one or more, preferably one of the aforementioned 5-HT-2A/C antagonists 2f, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment of the aforementioned disorders.

Another preferred embodiment of the invention is directed to a method for the treatment of one of the aforementioned disorders, comprising the administration of a therapeutically effective amount of one or more, preferably one of the aforementioned dopamine D4 antagonists 2g, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of a therapeutically effective amount of one or more, preferably one of the aforementioned dopamine D4 antagonists 2g, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment of the aforementioned disorders.

Another preferred embodiment of the invention is directed to a method for the treatment of one of the aforementioned disorders, comprising the administration of a therapeutically effective amount of one or more, preferably one of the aforementioned selective androgen receptor modulators (SARMs) 2h, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of a therapeutically effective amount of one or more, preferably one of the aforementioned selective androgen receptor modulators (SARMs) 2h, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment of the aforementioned disorders.

Another preferred embodiment of the invention is directed to a method for the treatment of one of the aforementioned disorders, comprising the administration of a therapeutically effective amount of one or more, preferably one of the aforementioned estrogens 2k, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of a therapeutically effective amount of one or more, preferably one of the aforementioned estrogens 2k, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment of the aforementioned disorders.

Another preferred embodiment of the invention is directed to a method for the treatment of one of the aforementioned disorders, comprising the administration of a therapeutically effective amount of one or more, preferably one of the aforementioned androgens 2l, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of a therapeutically effective amount of one or more, preferably one of the aforementioned androgens 2l, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment of the aforementioned disorders.

Another preferred embodiment of the invention is directed to a method for the treatment of one of the aforementioned disorders, comprising the administration of a therapeutically effective amount of one or more, preferably one of the aforementioned α-adrenergic receptor antagonist 2m, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of a therapeutically effective amount of one or more, preferably one of the aforementioned α-adrenergic receptor antagonist 2m, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment of the aforementioned disorders.

Another preferred embodiment of the invention is directed to a method for the treatment of one of the aforementioned disorders, comprising the administration of a therapeutically effective amount of one or more, preferably one of the aforementioned selective estrogen receptor modulators (SERMs) 2n, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of a therapeutically effective amount of one or more, preferably one of the aforementioned selective estrogen receptor modulators (SERMs) 2n, optionally in form of the pharmaceutically acceptable acid addition salts, in form of the hydrates and/or solvates and optionally in the form of the individual optical isomers, mixtures of the individual enantiomers or racemates thereof, for the preparation of a medicament for the treatment of the aforementioned disorders.

The present invention is further described in the following examples which are provided for illustrative purposes only and are not to be construed as limiting. Indeed, other variants of the invention will be readily apparent to one of ordinary skill in the art.

All publications and patents cited herein are incorporated by reference in their entireties.

The following examples demonstrate possible pharmaceutical compositions comprising flibanserin in combination with one of the aforementioned combination partners 2.

### EXAMPLE 1

### Combination 1 with 2c

| | |
|---|---|
| Constituents | mg/tablet |

Core

| | |
|---|---|
| Flibanserin (free base) | 50.000 |
| Sildenafil citrate | 70.225 |
| Anhydrous dibasic calcium phosphate | 100.000 |
| Microcrystalline cellulose | 203.090 |
| HPMC (Methocel E5) | 6.615 |
| Croscarmellose sodium | 8.820 |
| Magnesium stearate | 2.250 |

Coating

| | |
|---|---|
| HPMC (Methocel E5) | 4.320 |
| Polyethylene Glycol 6000 | 1.260 |
| Titanium dioxide | 1.800 |
| Talc | 1.542 |
| Iron oxide red | 0.078 |
| Total Film coated tablet | 450.000 |

| Constituents | mg/tablet |
|---|---|
| HPMC (Methocel E5) | 4.320 |
| Polyethylene Glycol 6000 | 1.260 |
| Titanium dioxide | 1.800 |
| Talc | 1.542 |
| Iron oxide red | 0.078 |
| Total Film coated tablet | 450.000 |

### EXAMPLE 2

### Combination 1 with 2d

| | |
|---|---|
| Constituents | mg/tablet |

Core

| | |
|---|---|
| Flibanserin (free base) | 50.000 |
| Aripiprazole | 10.000 |
| Lactose monohydrate | 133.750 |
| Microcrystalline cellulose | 40.000 |
| Hydroxypropylcellulose | 2.500 |
| Corn starch | 12.500 |
| Magnesium stearate | 1.250 |

Coating

| | |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide yellow | 0.043 |
| Total Film coated tablet | 255.000 |

### EXAMPLE 3

### Combination 1 with 2e

| | |
|---|---|
| Constituents | mg/tablet |

Core

| | |
|---|---|
| Flibanserin (free base) | 50.000 |
| Pramipexole dihydrochloride monohydrate | 1.000 |
| Lactose monohydrate | 143.490 |
| Microcrystalline cellulose | 47.810 |
| HPMC (e.g. Pharmacoat 606) | 2.500 |
| Carboxymethylcellulose sodium | 5.000 |
| Mannitol | 60.000 |
| Corn starch | 36.500 |
| Povidone | 1.000 |
| Colloidal silicon dioxide | 1.000 |
| Magnesium stearate | 1.700 |

Coating

| | |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated bilayer tablet | 357.000 |

### EXAMPLE 4

### Combination of 1 with 2f

| | |
|---|---|
| Constituents | mg/tablet |

Final Mixture

| | |
|---|---|
| Flibanserin (free base) | 50.000 |
| Ziprasidone hydrochloride monohydrate | 40.000 |
| Lactose monohydrate | 200.000 |
| Pregelatinized starch | 108.000 |
| Magnesium stearate | 2.000 |

Capsule

| | |
|---|---|
| Final Mixture | 400.000 |
| Capsule (size 1) | 82.000 |
| Total weight of Capsule | 482.000 |

The following examples show preferred pharmaceutical compositions of flibanserin, if the combinations according to the invention are administered in separate dosage units.

### EXAMPLE 5

### Composition

| | |
|---|---|
| Constituents | mg/tablet |

Core

| | |
|---|---|
| Flibanserin (free base) | 25.000 |
| Lactose monohydrate | 71.720 |
| Microcrystalline cellulose | 23.905 |
| HPMC (Methocel E5) | 1.250 |
| Carboxymethylcellulose sodium | 2.500 |
| Magnesium stearate | 0.625 |

Coating

| | |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 128.000 |

### EXAMPLE 6

### Composition

| | |
|---|---|
| Constituents | mg/tablet |

Core

| | |
|---|---|
| Flibanserin (free base) | 50.000 |
| Lactose monohydrate | 143.440 |
| Microcrystalline cellulose | 47.810 |
| HPMC (e.g. Pharmacoat 606) | 2.500 |
| Carboxymethylcellulose sodium | 5.000 |
| Magnesium stearate | 1.250 |

Coating

| | |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide red | 0.043 |
| Total Film coated tablet | 255.000 |

### EXAMPLE 7

### Composition

| | |
|---|---|
| Constituents | mg/tablet |

Core

| | |
|---|---|
| Flibanserin (free base) | 100.000 |
| Lactose monohydrate | 171.080 |
| Microcrystalline cellulose | 57.020 |
| HPMC (e.g. Methocel E5) | 3.400 |
| Carboxymethylcellulose sodium | 6.800 |
| Magnesium stearate | 1.700 |

Coating

| | |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated tablet | 347.000 |

### EXAMPLE 8

### Composition

| | |
|---|---|
| Constituents | mg/tablet |

Core

| | |
|---|---|
| Flibanserin (free base) | 2.000 |
| Dibasic Calciumphosphate, anhydrous | 61.010 |
| Microcrystalline cellulose | 61.010 |
| HPMC (Methocel E5) | 1.950 |
| Carboxymethylcellulose sodium | 2.600 |
| Colloidal silicon dioxide | 0.650 |
| Magnesium stearate | 0.780 |

Coating

| | |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 133.000 |

### EXAMPLE 9

### Composition

| | |
|---|---|
| Constituents | mg/tablet |

Core

| | |
|---|---|
| Flibanserin (free base) | 100.000 |
| Dibasic Calciumphosphate, anhydrous | 69.750 |
| Microcrystalline cellulose | 69.750 |
| HPMC (Methocel E5) | 2.750 |
| Carboxymethylcellulose sodium | 5.000 |
| Colloidal silicon dioxide | 1.250 |
| Magnesium stearate | 1.500 |

Coating

| | |
|---|---|
| HPMC (Methocel E5) | 2.400 |
| Polyethylene Glycol 6000 | 0.700 |
| Titanium dioxide | 1.043 |
| Talc | 0.857 |
| Total Film coated tablet | 255.000 |

### EXAMPLE 10

### Composition

| | |
|---|---|
| Constituents | mg/tablet |

Core

| | |
|---|---|
| Flibanserin (free base) | 20.000 |
| Lactose monohydrate | 130.000 |
| Microcrystalline cellulose | 43.100 |
| Hydroxypropyl Cellulose (e.g. Klucel LF) | 1.900 |
| Sodium Starch Glycolate | 4.000 |
| Magnesium stearate | 1.000 |

Coating

| | |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 205.000 |

1. A pharmaceutical composition comprising a therapeutically effective amount of flibanserin as one active ingredient in combination with a therapeutically effective amount of at least one additional active ingredient.

2. The pharmaceutical composition according to claim 1, wherein the additional active ingredient is selected from the group consisting of melanocortin agonists, prostaglandin E1 agonists, elevators of cyclic guanosine 3′,5′-monophosphate (cGMP), 5-HT-1A agonists, dopamine agonists, dopamine D4 antagonists, 5-HT-2A/C antagonists, selective androgen receptor modulators (SARMs), selective estrogen receptor modulators (SERMs), estrogens, androgens and α-adrenergic receptor antagonist.

3. The pharmaceutical composition according to claim 2, comprising a therapeutically effective amount of flibanserin and a therapeutically effective amount of one or more melanocortin agonist, optionally in combination with a pharmaceutically acceptable excipient.

4. The pharmaceutical composition according to claim 3, wherein the melanocortin agonist is selected from the group consisting of PT-141, MCL-0129, PG-917, and Ro-27-3225, or a pharmaceutically acceptable acid addition salt thereof, a hydrate and/or a solvate thereof, an optical isomer thereof, or a mixture of the enantiomers or a racemate thereof.

5. The pharmaceutical composition according to claim 2, comprising a therapeutically effective amount of flibanserin and a therapeutically effective amount of one or more prostaglandin E1 agonists, optionally in combination with a pharmaceutically acceptable excipient.

6. The pharmaceutical composition according to claim 5, wherein the prostaglandin E1 agonist, is selected from the group consisting of ornoprostil, limaprost, alprostadil, gemeprost, liprostin, NMI-775, prostaglandin E (PGE-1), papaverine, dioxyline, ethaverine, phentolamine, prazosin, minoxidil, nitroglycerin, alpha blockers, nitric oxide donors, and peptides, or a pharmaceutically acceptable acid addition salt thereof, a hydrate and/or a solvate thereof, an optical isomer thereof, or a mixture of the enantiomers or a racemate thereof.

7. The pharmaceutical composition according to claim 2, comprising a therapeutically effective amount of flibanserin and a therapeutically effective amount of one or more elevators of cGMP, optionally in combination with a pharmaceutically acceptable excipient.

8. The pharmaceutical composition according to claim 7, wherein the elevator of cGMP is selected from the group consisting of vardenafil, sildenafil, tadalafil, NCX-911, Sch-444877, FR-229934, 4-bromo-5-(pyridylmethylamino)-6-[3-(4-chlorophnyl)-propoxy]-3(2H)pyridazinone, 1-[4-(1,3-benzodioxol-5-ylmethyl)amiono]-6-chloro-2-[quinozolinyl]4-piperidine-carboxylic acid, monosodium salt, (+)-cis-5,6a,7,9,9,9a- hexahydro-2-[4-(trifluoromethyl)-phenylmethyl-5-methyl-cyclopent4,5]imidazo[2,1-b]purin-4(3H)one, furaziocillin, cis-2-hexyl-5-methyl-3,4,5,6a,7,8,9,9a-octahydrocyclopent[4,5]-imidazo[2,1-b]purin-4-one, 3-acetyl-1-(2-chlorobenzyl)-2 propylindole-6-carboxylate, 3-acetyl-1-(2-chlorobenzyl)-2-propylindole-6-carboxylate, 4-bromo-5-(3-pyridylmethylamino)-6-(3-(4-chlorophenyl) propoxy)-3-(2H)pyridazinone, 1-methyl-5(5-morpholinoacetyl-2-n-propoxyphenyl)-3-n-propyl-1,6-dihydro-7H-pyrazolo(4,3-d)pyrimidin-7-one, 1-[4-[(1,3-benzodioxol-5- ylmethyl)amino]-6-chloro-2-quinazolinyl}4-piperidinecarboxylic acid, monosodium salt, GF-196960, E-8010, E-4010, Bay-38-3045, Bay-38-9456, FR226807, Sch-51866, 5-(2-ethoxy-5-morpholinoacetylphenyl)-1-methyl-3-n-propyl-1,6-dihydro-7H-pyrazolo[4,3-d]pyrimidin-7-one, 3-ethyl-5-[5-(4-ethylpiperazin-1-ylsulfonyl)-2-n-propoxyphenyl]-2-(pyridin-2-yl)methyl-2,6-dihydro-7H-pyrazolo[4,3-d]pyrimidin-7-one, 3-ethyl-5-[5-(4-ethylpiperazin-1-ylsulfonyl)-2-(2methoxyethoxy)pyridin-3-yl]-2-(pyridin-2-yl)methyl-2,6-dihydro-7H-pyrazolo[4,3-d]pyrimidin-7-one, (+)-3-ethyl-5-[5-(4-ethylpieperazin-1-ylsulfonyl)-2-(2-methoxy-1(R)-methylethoxy)pyridin-3-yl]-2-methyl-2,6-dihydro-7H-pyrazolo[4,3-d]pyrimidin-7-one, 5-[2-ethoxy-5-(4-ethylpiperazin-1-ylsulfonyl)pyridin-3-yl]-3-ethyl-2-[2-methoxyethyl]-2,6-dihydro-7H-pyrazolo[4,3-d]pyrimidin-7-one, 5-[2-iso-butoxy-5-(4-ethylpiperazin-1-ylsulfonyl)pyridin-3-yl]-3-ethyl-2-(1-methylpiperidin-4-yl)-2,6-dihydro-7H-pyrazolo[4,3-d]pyrimidin-7-one, 5-[2-ethoxy-5-(4-ethylpiperazin-1-ylsulfonyl)pyridin-3-yl]-3-ethyl-2-phenyl-2,6-dihydro-7H-pyrazolo[4,3-d]pyrimidin-7-one, 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one, 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one, 4-(4-chlorobenzyl)amino-6,7,8-trimethoxyquinazoline, 7,8-dihydro-8-oxo-6-[2-propoxyphenyl]-1 H-imidazo[4,5-g]quinazoline, 1-[3-[1-[(4-fluorophenyl)methyl]-7,8-dihydro-8-oxo-1 H-imidazo[4,5-g]quinazolin-6-yl]-4-propoxyphenyl]carboxamide, 2-[2-ethoxy-5-(4-ethyl-piperazine-1-sulfonyl)-phenyl]-5-methyl-7-propyl-3H-imidazo[5,1-f]-[1,2,4]-triazin4-one, and 1-{6-ethoxy-5-[3-ethyl-6,7-dihydro-2-(2-methoxyethyl)-7-oxo-2H-pyrazolo[4,3-d]pyrimidin-5-yl]-3-pyridylsulfonyl}-4-ethylpiperazine, or a pharmaceutically acceptable acid addition salt thereof, a hydrate and/or a solvate thereof, an optical isomer thereof, or a mixture of the enantiomers or a racemate thereof.

9. The pharmaceutical composition according to claim 7, wherein the elevator of cGMP is selected from the compounds of formula 2c.1 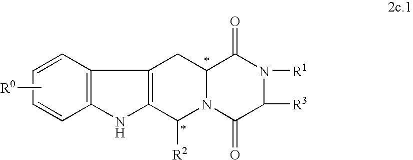
wherein
R⁰ represents hydrogen, halogen or C₁₋₆alkyl;
R¹ represents hydrogen, C₁₋₆alkyl, C₂₋₆alkenyl, C₂₋₆alkynyl, haloC₁₋₆alkyl, C₃₋₈cycloalkyl, C₃₋₈cycloalkyl-C₁₋₃alkyl, arylC₁₋₃alkyl or heteroaryl_{C1-3}alkyl;
R² represents an optionally substituted monocyclic aromatic ring selected from benzene, thiophene, furan and pyridine or an optionally substituted bicyclic ring 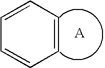
attached to the rest of the molecule via one of the benzene ring carbon atoms and wherein the fused ring A is a 5- or 6membered ring which may be saturated or partially or fully unsaturated and comprises carbon atoms and optionally one or two heteroatoms selected from oxygen, sulphur and nitrogen; and
R³ represents hydrogen or C₁₋₃alkyl, or R¹ and R³ together represent a 3- or 4-membered alkyl or alkenyl chain, or a pharmaceutically acceptable acid addition salt thereof, a hydrate and/or a solvate thereof, an optical isomer thereof, or a mixture of the enantiomers or a racemate thereof.

10. The pharmaceutical composition according to claim 7, wherein the elevator of cGMP is selected from the compounds of formula 2c.2 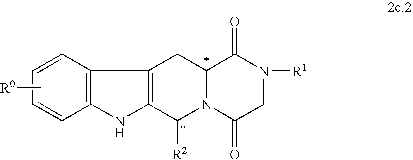
wherein
R⁰ represents hydrogen, halogen or C₁₋₆alkyl;
R¹ represents hydrogen,C₁₋₆alkyl, haloC₁₋₆alkyl, C₃₋₈cycloalkyl, C₃₋₈cycloalkyC₁₋₃alkyl, arylC₁₋₃alkyl or heteroarylC₁₋₃alkyl; and
R² represents an optionally substituted monocyclic aromatic ring selected from benzene, thiophene, furan and pyridine or an optionally substituted bicyclic ring 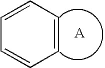
attached to the rest of the molecule via one of the benene ring carbon atoms and wherein the fused ring A is a 5- or 6-membered ring which may be saturated or partially or fully unsaturated and comprises carbon atoms and optionally one or two heteroatoms selected from oxygen, sulphur and nitrogen, or a pharmaceutically acceptable acid addition salt thereof, a hydrate and/or a solvate thereof, an optical isomer thereof, or a mixture of the enantiomers or a racemate thereof.

11. The pharmaceutical composition according to claim 2, comprising a therapeutically effective amount of flibanserin and a therapeutically effective amount of one or more 5-HT-1A agonists, optionally in combination with a pharmaceutically acceptable excipient.

12. The pharmaceutical composition according to claim 11, wherein the 5-HT-1A agonist is selected from the group consisting of Urapidil, Buspirone, Aripiprazole, Ziprasidone, Naftopidil, Tandospirone, Nemonapride, Gepirone, Repinotan, Sumanirole, Xaliproden hydrochloride, Bifeprunox, AP-521, SUN-N4057, Sarizotan, MKC-242, OPC-14523, Eptapirone maleate, SLV-308, BTS-79018, R-137696, F-13640, SSR-181507, SLV-314, SLV-319, 7-OH-DPAT, VN-2222, PD-158771, RS-30199, WAY-100012, A-74283, Enilospirone, Org-13011, B-8805-033, AP-159, AZ-16596, Anpirtoline, Ebalzotan, Binospirone, MDL-72832, RU-24969, Bay-r-1531, Ipsapirone, BIMG 80, BMS-181100, BMS-181101, BMS-181970, BMY-7378, BW-1205U90, B-20991, HAT-90B, Nerisopam, LY-175644, LY-178210, LY-228729, LY-274600, LY-274601, LY-293284, LY-301317, LY-315535, E-4414, E-6265 citrate, Lesopitron, RGH-1756, RGH-1757, 1192U90, HP-236, FG-5938, LEK-8804, LB-50016, RWJ-25730, EMD-56551, EMD-67478, EMD-77697, Roxindole, Vilazodone, BP-554, CGP-50281, CGS-12066B, CGS-18102, SDZ-MAR-327, CL-870801, CP-110330, CP-146662, CP-291952, FCE-23892, FG-5865, FG-5893, OSU-191, Sunepitron, U-67413B, U-86170, U-86192A, U-92016A, U-93385, Eptapirone, Mazapertine succinate, SL-870765, SL-880338, SR-59026, Bromerguride, Alnespirone, S-14506, S-14671, S-15535, S-15931, S-16924, S-213571, S-215521, Elopiprazole, Eltoprazine, Flesinoxan, Umespirone, SUN-8399, S-23751, PM-1000, LY 41, Adatanserin, WY-48723, Zalospirone and MDL-73975, or a pharmaceutically acceptable acid addition salt thereof, a hydrate and/or a solvate thereof, an optical isomer thereof, or a mixture of the enantiomers or a racemate thereof.

13. The pharmaceutical composition according to claim 2, comprising a therapeutically effective amount of flibanserin and a therapeutically effective amount of one or more dopamine agonists, optionally in combination with a pharmaceutically acceptable excipient.

14. The pharmaceutical composition according to claim 13, wherein the dopamine agonist is selected from the group consisting of ABT-724, CP-226269, bromocriptin, cabergolin, alpha-dihydroergocryptin, lisuride, pergolide, pramipexol, roxindol, ropinirol, sopirinol, talipexol, bupropion and terguride, or a pharmaceutically acceptable acid addition salt thereof, a hydrate and/or a solvate thereof, an optical isomer thereof, or a mixture of the enantiomers or a racemate thereof.

15. The pharmaceutical composition according to claim 2, comprising a therapeutically effective amount of flibanserin and a therapeutically effective amount of one or more 5-HT2A/2C antagonists, optionally in combination with a pharmaceutically acceptable excipient.

16. The pharmaceutical composition according to claim 15, wherein the 5-HT2A/2C antagonists is selected from the group consisting of Aripiprazole, Fluoxetine, Nefazodone, Pizotifen, Risperidone, Sarpogrelate, Ziprasidone, Agomelatine, Asenapine, Eplivanserin, Iloperidone, ketanserin, ritanserin, M 100907, Netamiftide, Ocaperidone, S-20098, Abaperidone, ACP-103, EMD 281014, EMR 62218, LU-31-130, SL 650472, EGIS-10037, LEK-8829, Nantenine, QF-2004B, R-107500, S 35120, S-14297, Amesergide, Amperozide, AT 1015, Balaperidone, BIMG 80, Deramciclane, EGIS 8465, EGIS 9933, Fananserin, FG 5803, FG 5893, FG-5938, FG-5974, GMC 1169, GMC 283, GMC 306, GMC 6139, ICI-169369, Irindalone, IT 657, JL-13, Lubazodone, LY 215840, LY-367265, NRA-0045, Org-38457, PNU-96415E, QF 0510B, QF 1003B, QF 1004B, RO 600946, Ro-60-0759, RP 71602, RS-102221, S 16924, S 213571, S 35031, S-17828, S-21357-1, SB 200646A, SB 206553, SB 221284, SB 228357, SB 242084, SB 243213, SDZ SER 082, TY 12283, TY-1 1223 and ZD-3638, or a pharmaceutically acceptable acid addition salt thereof, a hydrate and/or a solvate thereof, an optical isomer thereof, or a mixture of the enantiomers or a racemate thereof.

17. The pharmaceutical composition according to claim 2, comprising a therapeutically effective amount of flibanserin and a therapeutically effective amount of one or more dopamine D4 antagonists, optionally in combination with a pharmaceutically acceptable excipient.

18. The pharmaceutical composition according to claim 17, wherein the dopamine D4 antagonist is selected from the group consisting of olanzapine, ziprasidone, MDL-814608A, NRA-0562, S-18126, SPI-376, YM-50001, 1192U90, ALX-D4, Balaperidone, BIMG 80, Cl-1030, CP-293019, Fananserin, JL-13, L-741742, L-745870, L-751852, L-772620, L-800892, LU-35138, LUR-2366, NEO-376, NGB-4420, NGD-941, NRA-0045, NRA-0074, NRA-0154, NRA-0160, NRA-0161, NRA-0215, NRA-0219, NRA-0544, PD-089232, PD-108306, PD-165167, PD-167063, PD-168306, PD-172760, PD-172760, PD-172938, PD-35680, PD-82011, PNU-106161, PNU-106675, QF-1003B, QF-1004B, Ro-62-4599, S-16924, S-17828, Sch-71450, Sonepiprazole, U-101958, U-103073E, U-96415E and YM-43611, or a pharmaceutically acceptable acid addition salt thereof, a hydrate and/or a solvate thereof, an optical isomer thereof, or a mixture of the enantiomers or a racemate thereof.

19. The pharmaceutical composition according to claim 2, comprising a therapeutically effective amount of flibanserin and a therapeutically effective amount of one or more selective androgen receptor modulators (SARM), optionally in combination with a pharmaceutically acceptable excipient.

20. The pharmaceutical composition according to claim 19, wherein the selective androgen receptor modulator (SARM) is selected from the group consisting of LGD2226, LGD1331, bicalutamide, cyproterone acetate, hydroxyflutamide, spironolactone, 4-(trifluoromethyl)-2(1 H)-pyrrolidone[3,2-g]quinolinone, 1,2-dihydropyridono[5,6-g]quinoline and piperidino[3,2-g]quinolinone, or a pharmaceutically acceptable acid addition salt thereof, a hydrate and/or a solvate thereof, an optical isomer thereof, or a mixture of the enantiomers or a racemate thereof.

21. The pharmaceutical composition according to claim 2, comprising a therapeutically effective amount of flibanserin and a therapeutically effective amount of one or more estrogens, optionally in combination with a pharmaceutically acceptable excipient.

22. The pharmaceutical composition according to claim 21, wherein the estrogen is selected from the group consisting of estradiol (i.e. 1,3,5-estratriene-3,17β-diol, or “17β-estradiol”) 17α-estradiol, ethinylestradiol (i.e., 17α-ethinylestradiol), ethinylestradiol 3-acetate, ethinylestradiol 3-benzoate, estriol, estriol succinate, polyestrol phosphate, estrone, estrone acetate, estrone sulfate, piperazine estrone sulfate, quinestrol, and mestranol, or a pharmaceutically acceptable acid addition salt thereof, a hydrate and/or a solvate thereof, an optical isomer thereof, or a mixture of the enantiomers or a racemate thereof.

23. The pharmaceutical composition according to claim 2, comprising a therapeutically effective amount of flibanserin and a therapeutically effective amount of one or more androgens, optionally in combination with a pharmaceutically acceptable excipient.

24. The pharmaceutical composition according to claim 23, wherein the androgen is selected from the group consisting of including androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, ethylestrenol, oxandrolone, nandrolone phenpropionate, nandrolone decanoate, nandrolone furylpropionate, nandrolone cyclohexanepropionate, nandrolone benzoate, nandrolone cyclohexanecarboxylate, stanozolol, dromostanolone, dromostanolone propionate, testosterone, dehydroepiandrosterone (“prasterone”), sodium dehydroepiandrosterone sulfate, and 4-dihydrotestosterone (“stanolone” and 5α-dihydrotestosterone; the testosterone and 4-dihydrotestosterone esters formed from the hydroxyl group present at the C-17 position and the enanthate, propionate, cypionate, phenylacetate, acetate, isobutyrate, buciclate, heptanoate, decanoate, pentadecanoate, undecanoate, pelargonate, tridecanoate, palmitate, caprate, isocaprate, a-methylcaprate, β-methylcaprate, laurate, α-methylpelargonate, β-methylpelargonate, β, β-dimethylpelargonate, β-(p-methyl-cyclohexyl)propionate, β-(p-ethylcyclohexyl)-propionate, β-(cycloheptyl)-propionate, α-methyl-cyclohexyl-propionate, β-methyl-β-cyclohexyl-propionate, cyclododecyl-carboxylate, adamantine-1′-carboxylate, adamant-1′-yl-acetate, methyl-α-cyclohexyl propionate, and α-(bicyclo-[2,2,2-oct-1′-yl)-propionate, 3-n-hexylcyclobutanecarboxylate, 3-n-butylcyclopentanecarboxylate, 4-n-butylcyclohexanecarboxylate, 4-n-pentylcyclohexanecarboxylate and n-hexylcyclohexanecarboxylate; methyl testosterone, testolactone, oxymetholone, fluoxymesterone, or a pharmaceutically acceptable acid addition salt thereof, a hydrate and/or a solvate thereof, an optical isomer thereof, or a mixture of the enantiomers or a racemate thereof.

25. The pharmaceutical composition according to claim 2, comprising a therapeutically effective amount of flibanserin and a therapeutically effective amount of one or more α-adrenergic receptor antagonists, optionally in combination with a pharmaceutically acceptable excipient.

26. The pharmaceutical composition according to claim 25, wherein the α-adrenergic receptor antagonist is selected from the group consisting of phentolamine mesylate, HMP-12, REC-15/2615 and MPV 1248 (atipamezole), or a pharmaceutically acceptable acid addition salt thereof, a hydrate and/or a solvate thereof, an optical isomer thereof, or a mixture of the enantiomers or a racemate thereof.

27. The pharmaceutical composition according to claim 2, comprising a therapeutically effective amount of flibanserin and a therapeutically effective amount of one or more selective estrogen receptor modulators (SERM), optionally in combination with a pharmaceutically acceptable excipient.

28. The pharmaceutical composition according to claim 27, wherein the selective estrogen receptor modulator (SERM) is selected from the group consisting of tibolone, diethylstilbestrol, moxestrol, N-butyl-3,17-dihydroxy-N-methyl-estra-1,3,5(10)-triene-7-undecanamide (ICI 164,384), fulvestrant, raloxifene, trans-2,3-dihydroraloxifene, 4′ halo-raloxifene, 2-(alkyl raloxifene, 2-cycloalkyl raloxifene, 2-naphthyl raloxifene, 6-methoxy-2-(4-methoxyphenyl)-3-(4-nitrobenzoyl)-benzo[b]thiophene, arzoxifene, 2-(4-methoxyphenyl)-3-(4-(2-(1-piperidinyl)ethoxy)-phenoxybenzo(b)thiophene-6-ol); LY 117018 (6-hydroxy-2-(4-hydroxyphenyl)benzo(b)thien-3-yl)(4-(2-(1-pyrrolidinyl)ethoxy)phenyl)-methanone), bazedoxifen, idoxifene (1-[2-[4-(1 E)-1-(4-lodophenyl)-2-phenyl-1-butenyl]phenoxy]ethyl]pyrrolidine), droloxifene (3-[(1E)-1-[4-[2-(Dimethylamino)ethoxy]phenyl]-2-phenyl-1-butenyl]phenol), tamoxifen ((Z)-2-[4-(1,2-Diphenyl-1-butenyl)phenoxy]-N,N-dimethylethanamine), toremifene (2-[4-[(1 Z)4-Chloro-1,2-diphenyl-1-butenyl)phenoxy]-N,N-dimethylethanamine), clomiphene (2-[4-(2-Chloro-1,2-diphenylethenyl)phenoxy]-N,N-diethylethanamine), meproxifene ((4-(1-(4-(2-(dimethylamino)ethoxy)phenyl)-2-(4-(1-methylethyl)phenyl)-1-butenyl)-phenol), trioxifene, zindoxifene, lasofoxifene, nafoxidine, 3-[4-[1-(4-fluorophenyl)-2-phenyl-but-1-enyl]phenyl}acrylic acid, 3-[4-(1,2-diphenyl-but-1-enyl)-phenyl]-acrylic acid, cis-6-phenyl-5-(4-(2-pyrrolidin-1-yl-ethoxy)phenyl)-5,6,7,8-tetrahydronaphthalene-2-ol, cis-6-(4-fluorophenyl)-5-[4-(2-piperidin-1-yl-ethoxy)-phenyl]-5,6,7,8-tetrahydronaphthalene-2-ol, cis-1-[6′-pyrrolidinoethoxy-3′-pyridyl]-2-phenyl-6-hydroxy-1,2,3,4-tetrahydro-naphthalene, cis-6-(4′-hydroxyphenyl)-5-[4-(2-piperidin-1-yl-ethoxy)-phenyl]-5,6,7,8-tetrahydronaphthalen-2-ol, 6-(4-hydroxyphenyl)-5-[4-(2-piperidin-1-yl-ethoxy)-benzyl]-naphthalen-2-ol, 1-(4′-pyrrolidinoethoxyphenyl)-2-(4″-fluorophenyl)-6-hydroxy-1,2,3,4-tetrahydroisoquinoline, 1-(4′-pyrrolidinoethoxyphenyl)-2-phenyl-6-hydroxy-1,2,3,4 tetrahydroisoquinoline, or a pharmaceutically acceptable acid addition salt thereof, a hydrate and/or a solvate thereof, an optical isomer thereof, or a mixture of the enantiomers or a racemate thereof.

29. A method for the treatment of disorders selected from the group consisting of Hypoactive Sexual Desire Disorder, loss of sexual desire, lack of sexual desire, decreased sexual desire, inhibited sexual desire, loss of libido, libido disturbance, and frigidity, comprising administering a therapeutically effective amount of flibanserin, or a pharmaceutically acceptable acid addition salt thereof and/or a hydrate and/or a solvate thereof, in combination with a therapeutically effective amount of an additional active ingredient, or a pharmaceutically acceptable acid addition salt thereof, a hydrate and/or a solvate thereof, an optical isomer thereof, or a mixture of the enantiomers or a racemate thereof, separately or together within one pharmaceutical composition.

30. A method for the treatment of premenstrual disorders, comprising administering a therapeutically effective amount of flibanserin, or a pharmaceutically acceptable acid addition salt thereof and/or a hydrate and/or a solvate thereof, in combination with a therapeutically effective amount of an additional active ingredient, or a pharmaceutically acceptable acid addition salt thereof, a hydrate and/or a solvate thereof, an optical isomer thereof, or a mixture of the enantiomers or a racemate thereof, separately or together within one pharmaceutical composition.

31. A method for the treatment of sexual aversion disorder in females, comprising administering a therapeutically effective amount of flibanserin, or a pharmaceutically acceptable acid addition salt thereof and/or a hydrate and/or a solvate thereof, in combination with a therapeutically effective amount of an additional active ingredient, or a pharmaceutically acceptable acid addition salt thereof, a hydrate and/or a solvate thereof, an optical isomer thereof, or a mixture of the enantiomers or a racemate thereof, separately or together within one pharmaceutical composition.

32. A method for the treatment of sexual arousal disorder in females, comprising administering a therapeutically effective amount of flibanserin, or a pharmaceutically acceptable acid addition salt thereof and/or a hydrate and/or a solvate thereof, in combination with a therapeutically effective amount of an additional active ingredient, or a pharmaceutically acceptable acid addition salt thereof, a hydrate and/or a solvate thereof, an optical isomer thereof, or a mixture of the enantiomers or a racemate thereof, separately or together within one pharmaceutical composition.

33. A method for the treatment of orgasmic disorder in females, comprising administering a therapeutically effective amount of flibanserin, or a pharmaceutically acceptable acid addition salt thereof and/or a hydrate and/or a solvate thereof, in combination with a therapeutically effective amount of an additional active ingredient, or a pharmaceutically acceptable acid addition salt thereof, a hydrate and/or a solvate thereof, an optical isomer thereof, or a mixture of the enantiomers or a racemate thereof, separately or together within one pharmaceutical composition.

34. A method for the treatment of sexual pain disorders in females, comprising administering a therapeutically effective amount of flibanserin, or a pharmaceutically acceptable acid addition salt thereof and/or a hydrate and/or a solvate thereof, in combination with a therapeutically effective amount of and additional active ingredient, or a pharmaceutically acceptable acid addition salt thereof, a hydrate and/or a solvate thereof, an optical isomer thereof, or a mixture of the enantiomers or a racemate thereof, separately or together within one pharmaceutical composition.
